(19) United States
(12) Patent Application Publication
(10) Pub. No.: US 2014/0276027 A1
(43) Pub. Date: Sep. 18, 2014

(54) Devices, Systems, and Methods for Preservation of Arteriovenous Access Sites

(71) Applicant: Volcano Corporation, San Diego, CA (US)

(72) Inventors: Mary L. Gaddis, Newport Beach, CA (US); Ken Neeld, Charlotte, NC (US); Mark Turner, Indian Land, SC (US)

(73) Assignee: Volcano Corporation, San Diego, CA (US)

(21) Appl. No.: 14/212,696

(22) Filed: Mar. 14, 2014

Related U.S. Application Data
(60) Provisional application No. 61/794,665, filed on Mar. 15, 2013.

(51) Int. Cl. A61B 5/0215; A61B 5/00; A61B 8/12

(52) U.S. Cl. CPC A61B 5/0215; A61B 8/12; A61B 5/0066
USPC 600/427; 600/486; 600/459

(57) ABSTRACT

The present disclosure relates to devices, systems, and methods for evaluating and preserving arteriovenous access sites. More particularly, the present disclosure relates to a sensor wire that is sized, shaped, and configured to pass through a delivery instrument to measure pressure and flow within and around an AV access site, thereby indicating the impact of the arteriovenous access site on the blood flow to the surrounding vasculature and tissues in real time. Also, the present disclosure relates to a therapeutic system comprising a combination pressure-flow sensor wire, a balloon catheter with imaging capabilities, and a computer system to allow the user to evaluate the blood flow and blood pressure within and around an AV access site in real time, diagnose the presence of complications associated with arteriovenous access sites, treat such complications, and assess the effectiveness of treatment both during and after treatment.

### CROSS-REFERENCE TO RELATED APPLICATIONS

The present application claims priority to and the benefit of U.S. Provisional Patent Application No. 61/794,665, filed Mar. 15, 2013, which is hereby incorporated by reference in its entirety.

### BACKGROUND

End-stage renal disease (“ESRD”) is characterized by failure of the kidneys to properly excrete wastes, concentrate urine, and regulate electrolytes. In patients with ESRD, severe complications and death may result from the inappropriate accumulation of fluids and waste products in the body.

A common life-sustaining treatment for patients with ESRD is hemodialysis, which is a process whereby large volumes of blood are rapidly removed from the body and filtered through an extracorporeal machine that removes several waste products and excess fluids from the blood. The cleansed blood is then returned back into the body. In hemodialysis, three common devices are used to gain vascular access: an intravenous catheter, an arteriovenous (“AV”) fistula, and a synthetic AV graft. In catheter access, a dual lumen catheter may be inserted into a large vein to allow large volumes of blood to be withdrawn from one lumen, go through the dialysis machine, and be returned to the body through the other lumen. To create an AV fistula, a vascular surgeon joins an artery and a vein together via an anastomosis using the patient's own vessel, at least partially bypassing the capillary bed. Although AV grafts also involve the anastomosis of an artery and a vein, AV grafts utilize a prosthetic vessel to join the artery and vein.

The type of access chosen is influenced by factors such as the expected time or course of a patient's renal failure and the condition of his or her vasculature. Catheter access is rarely used for long-term dialysis due to the risk of complications including venous stenosis, thrombosis, and infection. AV grafts present the advantage of rapidly maturing grafts, but carry the risks of narrowing, thrombosis, and infection. AV fistulas are commonly recognized as a preferred method of access due to lower infection rates, higher blood flow rates, and a lower incidence of thrombosis.

One risk associated with AV access sites is the potential for the onset of vascular access steal syndrome or dialysis-associated steal syndrome (“DASS”), which describes vascular insufficiency resulting from the diversion of blood flow through a vascular access site. FIG. 1 illustrates a vascular system 10 including a vascular access site 15 connecting an artery 20 and a vein 25. If the blood flow rates through the AV access site 10 are too high and the collateral vasculature 30 that supplies the rest of the subject limb is insufficient, inordinate amounts of blood entering the subject limb may be drawn through the AV access site 15 and returned to the general circulation without entering the capillaries 35 of the subject limb. This vascular insufficiency may result in pallor, diminished pulses, decreased wrist-brachial index, decreased temperature, pain, and tissue damage of the limb distal to the AV access site 15. Another risk associated with AV access sites is thrombosis (and possible occlusion), which may result from inadequate rates of blood flow through the fistula or graft due to venous flow obstruction or stenosis.

The need exists for a device, system, and method to evaluate and address complications associated with vascular access sites such as, by way of non-limiting example, DASS and thrombosis. The devices, systems, and methods disclosed herein overcome one or more of the deficiencies of the prior art.

### SUMMARY

The present disclosure relates to devices, systems, and methods for evaluating and preserving arteriovenous access sites. More particularly, but not by way of limitation, the present disclosure relates to a sensor wire that is sized, shaped, and configured to pass through a delivery instrument to measure pressure and flow within and around an AV access site, thereby indicating the impact of the arteriovenous access site on the blood flow to the surrounding vasculature and tissues in real time. In addition, the present disclosure relates to a diagnostic system comprising a combination pressure-flow sensor wire, a delivery instrument, and a computer system to allow the user to evaluate the blood flow and blood pressure within and around an AV access site in real time (e.g., before, during, and after treatment). In some embodiments, the delivery instrument comprises an imaging catheter. In other embodiments, the delivery instrument comprises a delivery instrument such as a hollow-bore needle.

Also, the present disclosure relates to a therapeutic system comprising a combination pressure-flow sensor wire, a balloon catheter with imaging capabilities, and a computer system to allow the user to evaluate the blood flow and blood pressure within and around an AV access site in real time, diagnose the presence of complications associated with arteriovenous access sites, treat such complications, and assess the effectiveness of treatment both during and after treatment. Moreover, the present disclosure provides for a sensor wire that includes a protective sheath designed to prevent direct physical contact between the sensor wire and the patient, thereby allowing for the repeated use of the sensor wire in different patients. The devices, systems, and methods disclosed herein assess, record, and address the functionality of the AV access site, thereby enabling the user to diagnose and/or treat a variety of AV access related complications associated with dialysis, chemotherapy, and liver stenosis, such as, by way of non-limiting example, vascular stenosis, DASS, thrombosis, obstruction, occlusion, and infection.

It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTION OF THE DRAWINGS

The accompanying drawings illustrate embodiments of the devices and methods disclosed herein and together with the description, serve to explain the principles of the present disclosure.

FIG. 1 is a schematic diagram illustrating a conventional arteriovenous access site connecting an artery and a vein within a vascular system.

FIG. 2 is a schematic illustration of a diagnostic system according to one embodiment of the present disclosure.

FIG. 3 illustrates a partial cutaway side-view of a sensor wire coupled to a connector according to one embodiment of the present disclosure.

FIG. 4 illustrates a partial cutaway side-view of an elongated, flexible sensor wire coupled to a connector according to one embodiment of the present disclosure.

FIG. 5 illustrates a partial cutaway side-view of the sensor wire shown in FIG. 3 according to one embodiment of the present disclosure.

FIG. 6 illustrates a partial cutaway side-view of a sensor wire including a curved distal end according to one embodiment of the present disclosure.

FIG. 7 illustrates a partial cutaway side-view of a sensor wire including a moveable core wire according to one embodiment of the present disclosure.

FIG. 8 illustrates a partial cutaway side-view of the sensor wire shown in FIG. 5 at a different angle and positioned within a sheath according to one embodiment of the present disclosure.

FIG. 9 is a schematic representation of a partially cross-sectional side view of the delivery instrument advancing into an AV access site while the sensor wire remains outside the skin of a patient according to one embodiment of the present disclosure.

FIG. 10 is a schematic representation of a side view of the sensor wire encased in a sheath and disposed within the delivery instrument, wherein both the sensor wire and the delivery instrument are advancing into an AV access site of a patient according to one embodiment of the present disclosure.

FIG. 11 is a schematic illustration of a diagnostic and therapeutic imaging system according to one embodiment of the present disclosure.

FIG. 12 is a schematic illustration of a side view of a distal portion of the exemplary catheter shown in FIG. 11, including an exemplary marker coil according to one embodiment of the present disclosure.

FIG. 13 is a schematic illustration of the exemplary marker coil shown in FIG. 12.

FIG. 14 illustrates a partial cutaway side-view of an exemplary balloon catheter according to one embodiment of the present disclosure.

FIG. 15 is a diagrammatic illustration of a cross-sectional view of an AV access site connecting an artery and a vein.

FIGS. 16-21 show a method of inserting the delivery catheter and the sensor wire shown in FIG. 11 into an AV access site to evaluate the functionality of the AV access site according to one embodiment of the present disclosure.

FIG. 22 is a diagrammatic illustration of a cross-sectional view of the AV access site connecting an artery and a vein.

FIGS. 23-26 show a method of inserting the balloon catheter shown in FIG. 14 and the sensor wire shown in FIG. 11 into an AV access site to both evaluate the functionality of the AV access site and treat stenotic segments according to one embodiment of the present disclosure.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the disclosure is intended. Any alterations and further modifications to the described devices, instruments, methods, and any further application of the principles of the present disclosure are fully contemplated as would normally occur to one skilled in the art to which the disclosure relates. In particular, for the sake of brevity, the various embodiments of prosthetic devices and corresponding engagement structures are described below with reference to particular exemplary combinations of components, features, and structures. However, it is understood that the various components, features, and structures of the exemplary embodiments are combinable in numerous other ways. It is fully contemplated that the features, components, and/or steps described with respect to one embodiment may be combined with the features, components, and/or steps described with respect to other embodiments of the present disclosure. Thus, features from one embodiment may be combined with features from another embodiment to form yet another embodiment of a device, system, or method according to the present disclosure even though such a combination is not explicitly shown. Further, for the sake of simplicity, in some instances the same reference numbers are used throughout the drawings to refer to the same or like parts.

The various figures show embodiments of devices, systems, and methods suitable to assess and treat complications associated within an AV access site within a patient. As used herein, “AV access site” includes both an AV fistula and an AV graft. One of ordinary skill in the art, however, would understand that similar embodiments could be used to assess and improve the functionality of other vascular access sites without departing from the general intent or teachings of the present disclosure.

FIG. 2 illustrates a diagnostic system 100 according to one embodiment of the present disclosure. In the pictured embodiment, the diagnostic system 100 includes a sensor wire 110 slidably disposed within a delivery instrument 120, a patient interface module (“PIM”) 122, and a computer system 125. The delivery instrument 120 is shown in a cross-sectional view so that the sensor wire 110 can be seen inside the delivery instrument 120. In the pictured embodiment, the computer system 125 includes a processor 130, a memory 132, and an ultrasound pulse generator 135. In the pictured embodiment, the PIM includes a user input 138 and a display 140. The system 100 is arranged to facilitate the delivery of the sensor wire into an AV access site inside a patient's body, such as, by way of non-limiting example, the AV access site 15 shown in FIG. 1. The individual component parts of the diagnostic system 100 may be electrically, optically, and/or wirelessly connected to facilitate the transfer of power, signals, and/or data. The number and location of the components depicted in FIG. 2 are not intended to limit the present disclosure, and are merely provided to illustrate an environment in which the devices and methods described herein may be used.

In the illustrated embodiment, the sensor wire 110 is shaped and configured as an elongate, cylindrical tube. The sensor wire 110 includes a hollow elongate tube 145, a sensor assembly 148, and a core wire 155. In the pictured embodiment, the tube 145 is rigid. In other embodiments, as shown in FIG. 4, the tube is flexible. In one aspect, the core wire 155 extends between a proximal portion or connection assembly 160 and a distal portion 165 of the sensor wire 110. In the pictured embodiment, the sensor assembly 148 is coupled to the core wire 155 at the distal portion 165. The sensor assembly 148 may be attached to the core wire 155 or tube 145 in any of a variety of coupling mechanisms, including by way of non-limiting example, a snap-fit engagement, adhesive, welding, pressure fit, and/or mechanical fasteners. In the pictured embodiment, the sensor assembly 148 is attached to the core wire 155 via welding and a housing (not shown) around the sensor is bonded to the tube 145 via an adhesive. In a further embodiment, the sensor housing is directly attached to the elongate tube 145 and the core wire can be omitted, thereby forming a rigid sensor wire assembly. The sensor wire 110 and sensor assembly 148 will be described in further detail below with reference to FIGS. 5-7.

The sensor wire 110 is coupled to the computer system 125 in any of a variety of means known to those skilled in the art. In the pictured embodiment, the proximal portion 160 of the sensor wire 110 is coupled via a connector 170 to a supply cable 175 linked to the PIM 122, which is coupled to the computer system 125 via a supply cable 167. As noted above, the individual component parts of the diagnostic system 100 may alternatively or additionally be optically and/or wirelessly connected to facilitate the transfer of power, signals, and/or data. In some embodiments, the connector 170 and the PIM 122 are a single component (i.e., the connector 170 is the PIM 122). In some embodiments, the PIM 122 and the computer 125 are a single component.

In some embodiments, as shown in FIG. 3, the connector 170 has an inner passage 176 which can house the proximal portion 160 of the sensor wire 110. The sensor wire 110 may be selectively coupled to the connector 170 and the supply cable 175 in any of a variety of selective coupling mechanisms, including by way of non-limiting example, a threaded engagement, a snap-fit engagement, and a tension-based engagement. In some embodiments, the connector 170 comprises a handle sized such that it may be held and maneuvered by a user during a medical procedure. In the illustrated embodiment of FIG. 3, the connector is a conventional releasable connector utilized with coronary sensing systems sold by Volcano Corporation under the trade name ComboWire®. In some embodiments, the sensor wire 110 possesses sufficient column strength to support the weight of the connector 170 without causing damage to or deformation of the sensor wire 110. In some embodiments, the connector 170 can be disconnected to allow the advancement of a surgical instrument, such as, by way of non-limiting example, a balloon catheter, an irrigation catheter, an imaging catheter, another suitable surgical catheter, another sensor wire, or a guidewire, over the sensor wire 110 or in place of the sensor wire 110. In some instances, the sensor wire and the connector include similar features to and interact in ways similar to those disclosed for the guidewire and connector, respectively, in U.S. Pat. No. 8,231,537, entitled “Combination Sensor Guidewire and Methods of Use” and filed on Jun. 23, 2006, which is hereby incorporated by reference in its entirety.

With reference to FIG. 2, the delivery instrument 120 includes a lumen 178 extending between a distal end 180 and a proximal end 185. In the pictured embodiment, the distal end 180 of the delivery instrument 120 is shaped as a sharp distal tip configured to penetrate the skin, subcutaneous tissue, and other anatomic tissues of the patient (e.g., a vessel wall). In some embodiments, the delivery instrument 120 comprises a surgical needle. In other embodiments, the delivery instrument may comprise a surgical introducer or a catheter, which can be sized and shaped to allow the passage of the sensor wire 110 and/or other surgical devices from the proximal end 185 through the distal end 180. In some embodiments, the distal end 180 may be tapered to facilitate the entry and progress of the delivery instrument through tissues and/or vessels. In some embodiments, the delivery instrument may comprise the combination of a surgical introducer and either a surgical needle or a surgical catheter, wherein the introducer is sized and shaped to allow the passage of the needle or catheter from a proximal end through a distal end, the needle or catheter is inserted into a lumen of the introducer, and the sensor wire is inserted into a lumen of the needle or catheter.

The delivery instrument 120 may range in an outer diameter D1 from 1.9° F. (0.63 mm) to 4° F. (1.35 mm). A wall thickness T of the delivery instrument 120 may range from 0.001 to 0.005 inches. In one embodiment, the wall thickness T of the delivery instrument is 0.002 in (0.051 mm). In one embodiment, the delivery instrument 120 is a conventional 20 gauge surgical needle. In another embodiment, the delivery instrument is a conventional 22 gauge surgical needle. In another embodiment, the delivery instrument is a flexible needle capable of insertion into an AV access site (e.g., an AV fistula). In another embodiment, as described below with reference to FIG. 11, the delivery instrument is an imaging catheter, such as, by way of non-limiting example, the digital intravenous ultrasound (“IVUS”) catheter sold under the brand name of Eagle Eye® Platinum by Volcano Corporation of San Diego, Calif., or an optical coherence tomography (OCT) imaging catheter.

The sensor wire 110 extends through the lumen 178 of the delivery instrument 120. The sensor wire 110 is shaped such that it can be slidably disposed within the lumen 178, and the sensor wire 110 is sized such that the distal portion 165 can extend beyond the distal tip 180 of the delivery instrument 120. In other words, the sensor wire 110 is sized to be longer than the delivery instrument 120. In the pictured embodiment, the diameter of the sensor wire 120 is sized to be less than the diameter of the lumen 178 of the delivery instrument 120 to enable the sensor wire 110 to be reciprocally and axially moveable within the delivery instrument 120. In particular, the delivery instrument 120 and the sensor wire 110 are sized such that an outer diameter D2 of the sensor wire 110 is substantially equal to or less than an inner diameter D3 of the lumen 178 of the delivery instrument 120. This enables reciprocating movement of the sensor wire 110 along a longitudinal axis LA within the lumen 178 in directions designated by arrows 187 and 188.

The sensor wire 110 may range in diameter D2 from 0.014 in (0.356 mm) to 0.035 in (0.889 mm). For example, the sensor wire 110 may have any of a variety of diameters D2, including, by way of non-limiting example, 0.014 in (0.356 mm), 0.028 in (0.711 mm), and 0.035 in (0.889 mm). The delivery instrument 120 may have any of a variety of inner diameters D3, including, by way of non-limiting example, 0.010 in (0.254 mm). The delivery instrument 120 may range in length L from 40 cm to 120 cm. For example, the delivery instrument 120 may have any of a variety of lengths, including, by way of non-limiting example, 45 cm. With reference to FIG. 3, in some embodiments, the sensor wire 110 may range in length L2 from 40 to 60 mm. For example, the sensor wire 110 may have any of a variety of lengths, including, by way of non-limiting example, 40 cm.

In some instances, the sensor wire 110 may be entirely removed in the proximal direction from the delivery instrument 120. In other instances, the delivery instrument 120 may be entirely removed in the proximal direction from around the sensor wire 110. For example, in some embodiments, the connector 170 may be disconnected from the sensor wire 110 to allow the removal of the delivery instrument 120 in the proximal direction. When the user pierces the skin of a patient and advances the delivery instrument 120 in order to reach the target vessel, the delivery instrument 120 will pass through various neighboring tissues and fluids that may enter the lumen 178. In some embodiments, the outer diameter D2 of the sensor wire 110 closely approximates the inner diameter D3 of the lumen 178 of the delivery instrument 120, such that the sensor wire 110 can block undesired aspiration of bodily fluids and/or other substances into the lumen 178 of the delivery instrument 120 during a procedure. In instances where the outer diameter D2 of the sensor wire 110 is less than the inner diameter D3 of the lumen 178 of the delivery instrument 120, other means for blocking such undesired aspiration may be used. For example, in some embodiments, the delivery instrument includes a seal, such as, by way of non-limiting example, an O-ring, at the distal tip 180 to prevent or minimize the entry of such tissues and fluids into the lumen 178 as the delivery instrument is advanced to the target vessel. In some embodiments, the delivery instrument includes a conventional “bleed-back” chamber or valve. In some embodiments, the delivery instrument is coupled to a Tuohy-Borst adapter to prevent backflow of fluid during insertion into a patient.

In the pictured embodiment, the delivery instrument 120 includes a retaining feature 189 within the lumen 178 that prevents the sensor wire 110 from advancing a certain distance past the distal tip 180 and may selectively lock the sensor wire into position within the delivery instrument. In some instances, the retaining feature 189 extends circumferentially around the inner lumen 178. The retaining feature 189 may comprise any of a variety of retaining mechanisms, including, by way of non-limiting example, a flexible O-ring, a mechanical coupling, and or an adhesive such as “soft glue.” In some instances, the retaining feature 189 serves to center and/or align the sensor wire 110 with the distal tip 180 of the delivery instrument 120. Other embodiments may have any number of retaining features. Some embodiments lack a retaining feature.

The computer system 125 is configured for receiving, processing, and analyzing data in accordance with one embodiment of the present disclosure. In the pictured embodiment, the computer system 125 includes the processor 130, which is coupled to the memory 132, the ultrasound pulse generator 135, and the display 140. In some embodiments, the computer system 125 and the PIM 122 are integrated into a single device, such as, by way of non-limiting example, a compact user interface device including features of the SmartMap® Pressure Instrument sold by Volcano Corporation of San Diego, Calif.

The computer system 125 is coupled to the sensor wire 110, which carries the sensor assembly 148. In the pictured embodiment, the sensor assembly 148 includes a flow sensor 150 that comprises a Doppler ultrasound transducer. In some embodiments, the sensor assembly 148 may comprise an array of transducers. In some embodiments, the sensor assembly 148 comprises a plurality of sensors of the same or different types, including by way of non-limiting example, pressure, flow, temperature, and imaging. For example, in one embodiment, the sensor assembly 148 comprises a pressure sensor and a flow sensor. In such an embodiment, the pressure sensor may be located adjacent to the flow sensor or at a distance from the flow sensor. In some embodiments, the sensor wire 110 includes any combination of features possessed by the following guide wires sold by Volcano Corporation of San Diego, Calif.: the PrimeWire Prestige® PLUS Pressure Guide Wire, the FloWire® Doppler Guide Wire, and the ComboWire® XT.

The processor 130 may include one or more programmable processor units running programmable code instructions for implementing the methods described herein, among other functions. The processor 130 may be integrated within a computer and/or other types of processor-based devices suitable for a variety of medical applications. The processor 130 can receive input data from the sensor wire 110, the delivery instrument 120, and/or the ultrasound pulse generator 135 directly via wireless mechanisms or from wired connections such as the supply cable 175. The processor 130 may use such input data to generate control signals to control or direct the operation of the sensor wire 110, the delivery instrument 120, and/or the ultrasound pulse generator. In some embodiments, the user can program or direct the operation of the sensor wire 110, the ultrasound pulse generator 135, and/or the delivery instrument 120 from the user input 138. In some embodiments, the processor 130 is in direct wireless communication with the sensor wire 110, the ultrasound pulse generator 135, the delivery instrument 120, and/or the user input 138, and can receive data from and send commands to the sensor wire 110, the ultrasound pulse generator 135, the delivery instrument 120, and/or the user input 138.

In various embodiments, the processor 130 is a targeted device controller that may be connected to a power source (not shown) and/or accessory devices (such as, by way of non-limiting example, the display 140). In such a case, the processor 130 is in communication with and performs specific control functions targeted to a specific device or component of the system 100, such as the sensor wire 110 and/or the ultrasound pulse generator 135, without utilizing input from the user input 138. For example, the processor 130 may direct or program the sensor wire 110 and/or the ultrasound pulse generator 135 to function for a specified period of time, at a particular frequency, and/or at a particular angle of incidence without specific user input. In some embodiments, the processor 130 is programmable so that it can function to simultaneously control and communicate with more than one component of the system 100. In other embodiments, the system 100 includes more than one processor and each processor is a special purpose controller configured to control individual components of the system.

It should be appreciated that the processor 130 may exist as a single processor or multiple processor, capable of running single or multiple applications that may be locally stored in the processor 130 and/or memory 132 or remotely stored and accessed through the user input 138. It should also be appreciated that the memory 132 includes, but is not limited to, RAM, cache memory, flash memory, magnetic disks, optical disks, removable disks, and all other types of data storage devices and combinations thereof generally known to those skilled in the art

In the pictured embodiment, the processor 130 is configured to acquire Doppler ultrasound data from a blood vessel from the flow sensor 150 through the sensor wire 110, and can analyze the data to determine the presence or absence, the direction, and the amount of fluid flow (e.g., blood flow) in front of the delivery instrument 120. Doppler ultrasound measures the movement of objects through the emitted beam as a phase change in the received signal. When ultrasound waves are reflected from a moving structure (e.g., a red blood cell within a vessel), the wavelength and the frequency of the returning waves are shifted. If the moving structure is moving toward the transducer, the frequency increases. If the moving structure is moving away from the transducer, the frequency decreases. In some embodiments, the processor 130 employs the Doppler Equation Δf=(2f₀V Cos θ)/C, where Δf is the frequency shift, f₀ is the frequency of the transmitted wave, V is the velocity of the reflecting object (e.g., a red blood cell), θ is the angle between the incident wave and the direction of the movement of the reflecting object (i.e., the angle of incidence), and C is the velocity of sound in the medium. The frequency shift is maximal if the sensor 150 is oriented parallel to the direction of the blood flow and the θ is zero degrees (cos 0=1). The frequency shift is absent if the sensor 150 is oriented perpendicular to the direction of the blood flow and the θ is 90 degrees (cos 90=0). Higher Doppler frequency shifts are obtained the velocity is increased, the incident wave is more aligned to the direction of blood flow, and/or if a higher frequency is emitted. In other embodiments, the sensor 150 may comprise a different type of flow sensor.

In the pictured embodiment, the processor 130 is connected to the ultrasound pulse generator 135, and may control the ultrasound pulse generator. The ultrasound pulse generator 135 may comprise an ultrasound excitation or waveform generator that provides control signals (e.g., in the form of electric pulses) to the sensor wire 110 to control the ultrasound wave output from the sensor 150. In some instances, the ultrasound pulse generator 135 directs continuous wave ultrasound from the sensor 150, instead of pulsed wave ultrasound. In some instances, the ultrasound generator 135 is part of the processor 130. In other instances, the ultrasound generator 135 is integrated in the sensor wire 110.

In the pictured embodiment, the processor 130 is connected to the display 140, which is configured to convey information, including for example blood pressure and/or flow data gathered from the sensor wire 110, to the user. In some instances, the processor 130 creates an appropriate indication to display via the indicating apparatus 140. In some instances, the display 140 may be an oscillator or an auditory device configured to convey information to the user via auditory methods, such as meaningful tonality to convey different information. In other instances, the display 140 may convey information via tactile sensations, including by way of non-limiting example, increasing vibration to reflect an increase in blood pressure or an increase in flow rate. In other instances, the display 140 may comprise a visual display configured to graphically display the measured data to the user. In some embodiments, the data received from the sensor wire 110 and/or the delivery instrument 120 may be stored in the memory 132 and accessed by the processor for visual depiction on the display 140. For example, in one embodiment, the display 140 may graphically depict the average or individual flow rates measured through the AV access site over a selected or predetermined period of time.

In some embodiments, the Doppler shift information is displayed in wave form. In some embodiments, the Doppler shift information is displayed as color information superimposed on a background gray scale B mode ultrasound image. In some embodiments, a positive Doppler shift is assigned one color and a negative Doppler shift is assigned another color. In some embodiments, the magnitude of the Doppler shift is represented by the different gradients of brightness of the assigned color. In some embodiments, the intravascular pressure and flow measurements are simultaneously depicted on the display 140. In some embodiments, the display 140 includes similar features to the ComboMap® Pressure and Flow System sold by Volcano Corporation of San Diego, Calif.

Referring to FIG. 3, the connector 170 is illustrated attached to the sensor wire 110. The connector 170 has a length L3. In one embodiment, L3 is about 5-15 cm in length. In still a further embodiment, L3 is 8-10 cm in length. The connector can range in length and orientation. As mentioned above, in some embodiments, the connector 170 and the PIM 122 are a single component. In such embodiments, the connector 170 is shaped and sized to be compact enough to facilitate the ease of use, transport, and setup of the system. In some embodiments, the connector 170 is shaped and sized to permit convenient setup at a small workstation (e.g., a workstation within an outpatient facility), as well as mounting on an intravenous pole or at a patient's bedside.

FIG. 4 illustrates an intravascular sensor wire 200 connected to the connector assembly 170 of the sensing system. The sensor assembly 202 is substantially identical in characteristics to the sensor wire 110 except for the differences noted herein. In particular, the sensor wire 200 includes a flexible elongate tube 201. The sensor wire 200 includes a distal sensor assembly 202 positioned adjacent a distal end 203. The distal sensor assembly 202 can include one or more sensors such as pressure, flow, temperature, and/or imaging. The sensor assembly 202 is substantially identical in characteristics to the sensor assembly 148. A communication connection assembly 204 on a proximal portion 206 is configured to substantially match the outer diameter and length of a communication connection assembly (e.g., communication connection assembly 230 shown in FIG. 5) of the shorter access sensor wire 110. In one embodiment, the two connection assemblies are identical in the number of electrical connectors, the diameter of the connectors, and their axial spacing along the axis. In this form, both sensor wires may be sequentially received within the female lumen 176 of the connector 170. It is contemplated that while the different sensor wires 200, 110 may include a different number of conductive bands, the spacing between the bands must match the spacing of electrical contacts within the connector lumen 176. The sensor wire 200 is a very flexible wire suitable for passing through a tortuous vascular route. In one embodiment, the sensor wire 200 is shaped and sized for passage through a rigid, shorter, needle-like delivery instrument 120. In other embodiments, the sensor wire 200 is shaped and sized for passage through a flexible, elongated, catheter-like delivery instrument 120. In some embodiments, the sensor wire 200 is shaped and sized for passage through both types of delivery instrument. In some embodiments, the sensor wire has a length ranging 40-60 cm. In some embodiments, the sensor wire length will be at least 10 times the length L3 of the connector 170.

In one embodiment, after the delivery instrument 120 has been positioned within the AV access site, the distal end 203 of the elongated sensor wire 200 can be passed through the delivery instrument into the AV access site. The elongated sensor wire 200 can then be advanced from the initial AV access segment into other vessel segments of the vasculature of the patient. The proximal connection assembly 204 can then be inserted into the lumen 176 of the connector 170 and the distal barrel of the connector rotated to lock the connection assembly in place. The sensing system can be utilized in a conventional fashion with the computer system for receiving signals, analyzing the signals, and providing an output to the user based on the sensed signals. Depending on the type of sensor assembly 202, the intravascular sensor assembly can detect pressure, flow, temperature, or image the AV access site or vessel segment spaced up to the length of the sensor wire 200 away from the delivery instrument.

FIG. 5 illustrates a partial cutaway side-view of the sensor wire 110 according to one embodiment of the present disclosure. The sensor wire 110 comprises the elongate tube 145 and the sensor assembly 148. In the pictured embodiment, the sensor assembly 148 includes the pressure sensor 220 and the flow sensor 150. The pressure sensor 220 can be used to sense the pressure of blood within the AV access site and neighboring blood vessels. As mentioned above, in the pictured embodiment, the flow sensor 150 comprises an ultrasound transducer configured to emit ultrasound waves and receive reflected ultrasound waves. In other embodiments, the sensor may comprise a separate ultrasound transmitter and receiver, wherein the transmitter and receiver may be communicatively coupled to each other via either a wired or wireless link. In the pictured embodiment, the sensor is shown as a single transducer. In alternative embodiments, the sensor may be any number of transducers, shaped in any of a variety of shapes and arranged in any of a variety of arrangements. In some embodiments, the sensor (and/or the sensor wire 110) includes additional amplifiers to achieve the desired sensitivity to the nature of the target fluid flow (e.g., blood flow and/or heart rate). It should also be appreciated that the sensor depicted herein is not limited to any particular type of sensor, and includes all Doppler sensors and/or ultrasonic transducers known to those skilled in the art. For example, a sensor wire having a single transducer adapted for rotation or oscillation, as well as a sensor wire having an array of transducers circumferentially positioned around the sensor wire are both within the spirit and scope of the present invention. In addition, the sensor may include an optical sensor and/or an imaging sensor.

In the pictured embodiment, the elongate tube 145 is shaped as a rigid, hollow cylinder having a lumen 222 with a circular cross-sectional shape. In various embodiments, the elongate tube 145 can have any of a variety of cross-sectional shapes, including, for example, rectangular, square, or ovoid. The lumen 222 is shaped and sized to receive the core wire 155 and various electrical conductors 192 extending from the sensor assembly 148. The illustrated embodiment includes conductors extending to the pressure sensor 220 and conductors extending from the ultrasound transducer 150 to the ultrasound energy supply (e.g., the supply cable 175 and the ultrasound pulse generator 135 (shown in FIG. 2)).

Also depicted in the pictured embodiment are conductive bands 224 positioned at the proximal portion 160 of the sensor wire 110 forming a communication connection assembly 230. Various embodiments may include any number and arrangement of electrical conductors and conductive bands. Other embodiments may lack electrical conductors 192 and/or the conductive bands 193.

Within the tube 145, the sensor assembly 148, including the ultrasound sensor 210, is maintained in substantial alignment with the communication connection assembly 230 during use. In some embodiments, the strength of the rigid elongate tube 145 is sufficient to hold the weight of the female connector 170 along with the associated cable 175 without substantially yielding from the longitudinal axis. However, in alternative embodiments, as shown in FIG. 4, the elongate tube may be semi-rigid and partially flexible and allow the connection assembly to be longitudinally offset from the sensor assembly.

As illustrated in FIG. 5, the connection assembly 230 has a substantially uniform diameter with each conductive band 224 axially spaced coaxially along the longitudinal axis with matching outer diameters. The outer diameter of the connection assembly 230 substantially matches the outer diameter of the elongated tube 145 and the sensor assembly 148. Thus, the sensor wire 110 has a uniform outer diameter along its entire length. In addition to the alternatives set forth above, the outer diameter may be 0.028 or 0.035 inches in two alternative embodiments.

The elongate tube 145 may be composed of any of a variety of suitable biocompatible materials that are able to provide the desired amount of strength, rigidity, and corrosion resistance, including, by way of non-limiting example, Nitinol, stainless steel, titanium, nickel titanium alloys, cobalt alloys, combinations of tungsten/gold with stainless steel or cobalt alloys, alloys thereof, and polymers such as polyimide, polyetheretherketone (PEEK), polyamide, polyetherblockamide, polyethylene, polytetrafluoroethylene (PTFE), fluorinated ethylene propylene (FEP), and polyurethane. In some instances, as mentioned above, the elongate tube 145 possesses sufficient column strength and resilience to support the weight of the connector 170 (shown in FIGS. 2 and 3) without causing damage to or deformation of the sensor wire 110. In the pictured embodiment, the elongate tube 145 possesses a substantially constant degree of stiffness along its length. In some instances, the sensor wire 110 has varying stiffness and flexibility along its length due to changes in material composition, thickness, and cross-sectional shape of the elongate tube 145.

An outer wall 240 of the elongate tube 145 may have any of a variety of thicknesses, including, by way of non-limiting example, 0.002 inches (0.051 mm). For example, the outer wall 240 may range in thickness from 1 mm to 40 mm. In some embodiments, the outer wall 240 may be treated or coated with a material to give the sensor wire 110 a smooth outer surface with low friction. In some embodiments, the sensor wire 110 is coated with a material along its length to ease insertion through the lumen 178 of the delivery instrument 120. For example, the entire length of sensor wire 110 or a portion of its length may be coated with a material that has lubricating or smoothing properties. Exemplary coatings can be hydrophobic or hydrophilic. Typical coatings may be formed from, by way of non-limiting example, polytetraflouroethylene (PTFE) or Teflon™, a silicone fluid, or urethane-based polymers. Additionally or alternatively, other biocompatible coatings that provide the above mentioned properties could be used.

In certain embodiments, the sensor wire 110 may include radioopaque markers. For example, in the embodiment shown in FIG. 5, the outer wall 240 includes three radioopaque markers 242 coupled to the outer wall 240 and three radiopaque markers 244 coupled to an imagable portion of the core wire 155. The radioopaque markers 242, 244 comprise tubular markers that circumferentially surround the sensor wire 110 and the core wire 155, respectively. In other embodiments, the radioopaque markers may be shaped and configured in any of a variety of suitable shapes, including, by way of non-limiting example, rectangular, triangular, ovoid, linear, and non-circumferential shapes. The radiopaque markers 250 may be formed of any of a variety of biocompatible radioopaque materials that are sufficiently visible under fluoroscopy to assist in the transseptal procedure. The radioopaque markers 250 permit the physician to fluoroscopically visualize their location and orientation within the patient. For example, when a portion of the imagable section extends into the AV access site, X-ray imaging of the radioopaque markers 242, 244 may confirm successful insertion into the AV access site, neighboring vessels, and/or collaterals. Such radiopaque materials may be fabricated from, by way of non-limiting example, platinum, gold, silver, platinum/iridium alloy, and tungsten. The markers 242, 244 may be attached to the sensor wire 110 using a variety of known methods such as adhesive bonding, lamination between two layers of polymers, or vapor deposition, for example. Various embodiments may include any number and arrangement of radiopaque markers. In some embodiments, the sensor wire lacks radiopaque markers.

With reference to FIG. 5, a distal end 250 of the sensor wire 110 including the ultrasound transducer 150 is shaped and configured as a blunt, atraumatic tip. In the pictured embodiment, the distal end 250 is shaped as a straight tube terminating in a rounded, hemispherical dome. In other embodiments, the distal end may have any of a variety of atraumatic shapes, provided that the distal end is configured to not penetrate tissue in the absence of undue pressure. In some embodiments, the distal end 250 may be sufficiently malleable and flexible to eliminate the need for the curve of the tip to be atraumatic. In some embodiments where penetration of tissue by the sensor wire 110 is desired, the distal end can be sharp and/or have angular edges. In the pictured embodiment, the ultrasound transducer 150 (i.e., the flow sensor) is shaped and configured to convey ultrasound energy along the longitudinal axis of the device through the distal end 250.

FIG. 6 illustrates a distal end 255 of a sensor wire 260, which is substantially identical to the sensor wire 110 except for the differences described herein. The distal end 255 is shaped as a curved tube terminating in a rounded, hemispherical dome. In some embodiments, a distal tip 265 may be open to allow for the passage of the core wire 155 and the sensor assembly 148 through the distal tip. In some embodiments, the distal end 255 and/or the core wire 155 may be constructed from a structurally deformable biocompatible material that can elastically or plastically deform without compromising its integrity. The distal end 255 and/or the core wire 155 may be made from a self-expanding biocompatible material, such as Nitinol or a resilient polymer, or an elastically compressed spring temper biocompatible material. Other materials having shape memory characteristics, such as particular metal alloys, may also be used. The shape memory materials allow the distal end 255 and the core wire 155 to be restrained in a low profile configuration during delivery into the AV access site and to resume and maintain its curved shape in vivo after the delivery process. In some embodiments, the material composition of the core wire 155 resiliently biases the distal end 255 toward the curved condition.

In particular, in this example, the core wire 155 is formed of an elastic material allowing the distal end 255 to elastically deform to a straight state to facilitate delivery through a tubular delivery instrument 120, and spring back to a curved state as it enters the AV access site and/or vessel. In other embodiments, the core wire 155 may be made of a shape memory alloy having a memory shape in the curved configuration. The shape memory materials may help to prevent the sensor wire 260 from kinking or buckling during use within the AV access site and/or vessels. In some embodiments, the core wire 155 and/or the distal end 255 may curve into a configuration that correlates with a typical angle found at an AV access anastomosis site, linking an AV fistula or AV graft to a patient's native blood vessels. Such a configuration may facilitate the passage of the sensor assembly into different areas within and around the AV access site to enable atraumatic and efficient assessment of the functionality of the AV access site. Though the distal end 255 in the pictured embodiment curves into a J-shape or hook shape, the end section may be configured to curve into any of a variety of shapes, such as, by way of non-limiting example, an oval, a loop, and a helix.

FIG. 7 illustrates a sensor wire 300, which is substantially identical to the sensor wire 110 except for the differences described herein. The sensor wire 300 includes a core wire 305, which is substantially similar to the core wire 155 except for the differences described herein, that carries the sensor assembly 148 within an elongate tube 310, which is substantially similar to the elongate tube 145 except for the differences described herein. The outer diameter of the core wire 305 and a height H of the sensor assembly 148 are sized to be less than the diameter of a lumen 315 of the elongate tube 310 to enable the core wire 305 to be reciprocally and axially moveable within the lumen 315. In particular, the elongate tube 310, the sensor assembly 148, and the core wire 305 are sized such that the height H of the sensor wire 110 is substantially equal to or less than an inner diameter D4 of the lumen 315 of the elongate tube 310. This enables reciprocating movement of the core wire 315 and the sensor assembly 148 along a longitudinal axis LA within the lumen 315 in directions designated by arrows 320 and 322. The elongate tube 310 includes an open distal end 325, thereby permitting the sensor assembly 148 to emerge from the distal end 325 of the sensor wire 300 into direct contact with the contents of an AV access site and/or blood vessel.

In some embodiments, the core wire 305 is coated with a material along its length to ease movement through the lumen 325. For example, the entire length of core wire 305 or a portion of its length may be coated with a material that has lubricating or smoothing properties. Exemplary coatings can be hydrophobic or hydrophilic. Typical coatings may be formed from, by way of non-limiting example, polytetraflouroethylene (PTFE) or Teflon™, a silicone fluid, or urethane-based polymers. Additionally or alternatively, other biocompatible coatings that provide the above mentioned properties could be used.

In FIG. 8, the sensor wire 110 is shown partially surrounded or encased by a sheath 350. In some embodiments, the sensor wire 110 can be disposable in order to prevent the transfer of contagious diseases among different patients. In other embodiments, however, the sensor wire 110 may be reusable for performing medical procedures on different patients. If used with the sheath 350, for example, the sensor wire 110 can be reused on different patients because the probability of transferring a virus or bacterium among patients is reduced through the use of a disposable barrier such as the sheath 350. In other instances, the sensor wire 110 may be reused for procedures on different patients if it is sterilized between procedures. In some embodiments, the sheath 350 includes features of the sheath disclosed in U.S. Provisional Patent Application No. 61/737,040, entitled “Devices, System, and Methods for Targeted Cannulation,” filed on Dec. 13, 2012 with inventor Stigall, incorporated herein by reference in its entirety.

In the pictured embodiment, the elongated, flexible, protective sheath 350 extends from a proximal end 355 to a distal end 360. The proximal end 355 is open and relatively larger in diameter than the closed distal end 360. In the pictured embodiment, the sheath 350 is transparent, and, in particular, transparent to ultrasound energy. In the pictured embodiment, the inner diameter D5 of the sheath 350 is slightly larger than the outer diameter D2 of the sensor wire 110 (shown in FIG. 2). An outer diameter D6 of the sheath 350 is slightly smaller than the inner luminal diameter D3 of the delivery instrument 120 (shown in FIG. 2). Thus, the sensor wire 110, even when encased within the sheath 350, can move back and forth along the longitudinal axis LA within the lumen 178 of the delivery instrument 120 (shown in FIG. 2).

FIG. 9 illustrates a partially cross-sectional side view of the delivery instrument 120 advancing into an AV access site 400 while the sensor wire 110 remains outside the skin S according to one embodiment of the present disclosure. In the pictured embodiment, the delivery instrument 120 comprises a hollow bore needle. Once the delivery instrument 120 is optimally positioned to penetrate the AV access site 400, the user can advance the delivery instrument 120 through the skin S and into the AV access site. Actual penetration of the AV access site 400 may be indicated by back flow of the blood into the delivery instrument 120 and/or a bleedback chamber or valve. In the pictured embodiment, the sensor wire 110 remains at the skin surface as the delivery instrument 120 is advanced into the AV access site 400. In some embodiments, the user may manually prevent the sensor wire 110 from advancing with the delivery instrument 120 by holding the sensor wire 110 in place proximal to the delivery instrument 120 (e.g., by the connector 170 shown in FIG. 2). In other embodiments, the sensor wire 110 may be temporarily restrained within the delivery instrument by the connector 170 or by the retaining feature 189 within the lumen 178 of the delivery instrument 120 (shown in FIG. 2).

FIG. 10 is a schematic representation of a side view of the sensor wire 110 encased in the sheath 350 and disposed within the delivery instrument 120, wherein both the sensor wire and the delivery instrument are advanced into the vessel V according to one embodiment of the present disclosure. As mentioned above, the distal end 250 of the sensor wire 110 is shaped and configured to emerge from the distal tip 180 of the delivery instrument 120 into the AV access site 400. Once the sensor wire 110 is positioned within the AV access site 400 (and/or neighboring vasculature), the user can activate the sensor assembly 148 and begin processing pressure, flow, and/or imaging data received by the sensor assembly. The reflected signals obtained by the sensor assembly 148 are communicated to the processor 130, which conveys the reflected data to the display 140 (shown in FIG. 2).

In this instance, the sensor wire 110 is inserted into the sheath 350 before being inserted into the delivery instrument 120. The user can advance the sensor wire 110 and sheath 350 along with the delivery instrument 120 into the AV access site 400 (and/or neighboring vasculature) without contaminating the sensor wire 110 (i.e., because the sheath 350 shields the sensor wire 110 from any tissue and fluid encountered within the patient). Actual penetration of an AV access site (and/or neighboring vasculature) may be indicated by back flow of the blood into the delivery instrument 120 and/or a bleedback chamber or valve.

In one exemplary method, the user may sequentially insert the delivery instrument 120 into the AV access site and then into neighboring vessels and/or collaterals in order to assess the functionality of the AV site and to assess for complications such as, by way of non-limiting example, DASS (particularly in the case of AV fistulas), stenosis, thrombosis, and infection. In other instances, the user may sequentially insert the delivery instrument 120 into the AV access site and the neighboring vessels and/or collaterals in any order or sequence in order to assess for these complications. This method of assessing AV access-related complications is further described below in relation to FIGS. 15-26.

FIG. 11 is a schematic illustration of a diagnostic and therapeutic imaging system 500 according to one embodiment of the present disclosure. In the pictured embodiment, the system 500 includes a sensor wire 510 slidably disposed within a delivery catheter 515, a patient interface module (“PIM”) 122, an IVUS display 520, an IVUS console 525, and a computer system 125. The sensor wire 510 is substantially identical to the sensor wire 200 shown in FIG. 4 except for the differences noted herein. The delivery catheter is substantially identical to the delivery instrument 120 shown in FIG. 2 except for the differences noted herein. The individual component parts of the system 500 may be electrically, optically, and/or wirelessly connected to facilitate the transfer of power, signals, and/or data. The number and location of the components depicted in FIG. 11 are not intended to limit the present disclosure, and are merely provided to illustrate an environment in which the devices and methods described herein may be used.

The system 500 is capable of the diagnostic procedures of the system 100, as well as receiving, processing, and analyzing IVUS images in accordance with one embodiment of the present disclosure. In the pictured embodiment, the delivery catheter 515 comprises a flexible IVUS catheter sized and shaped to allow the passage of the sensor wire 510 within a lumen 530. The delivery catheter 515 is shown in a cross-sectional view so that the sensor wire 510 can be seen inside the lumen 530, which extends from a proximal end 532 to a distal end 534 of the imaging catheter 515. The delivery catheter 515 includes an imaging device, such as, by way of non-limiting example, an IVUS transducer 540, at the distal end. The IVUS console 525, which can acquire RF backscattered data (i.e., IVUS data) from an AV access site and/or blood vessel through the delivery catheter 515, is connected to the PIM 122, the IVUS display monitor 520, and the computer device 125. It should be appreciated that the IVUS console 525 depicted herein is not limited to any particular type of IVUS console, and includes all ultrasonic devices known to those skilled in the art. For example, in one embodiment, the IVUS console 525 may be a Volcano S5™ Imaging System. In other embodiments, the IVUS console 525 is replaced by an optical coherence tomography (OCT) console and the delivery catheter 515 includes an OCT imaging element.

In general, the catheter 515 is sized and shaped for use within an internal structure of a patient, including but not limited to a patient's AV access site, arteries, veins, heart chambers, neurovascular structures, gastrointestinal system, pulmonary system, and/or other areas where internal access of patient anatomy is desirable. In that regard, depending on the particular medical application, the catheter 515 is configured for use in cardiology procedures, neurovascular procedures, pulmonary procedures, endoscopy procedures, colonoscopy procedures, and/or other medical procedures.

The lumen 530 is shaped and configured to allow the passage of fluid, cellular material, or another medical device (e.g., a guidewire) from the proximal end 532 to the distal end 534. In the pictured embodiment, the lumen 530 is sized to accommodate the reciprocal motion of the sensor wire 510. In some embodiments, the lumen 530 is sized to accommodate the passage of a conventional guidewire. In such an embodiment, the lumen 530 has an internal diameter greater than 0.014 inches.

The distal end 534 is configured to be inserted into a body cavity, tissue, or tubular organ system of a patient. In some embodiments, the distal end 534 is tapered to facilitate insertion of the catheter into a patient. In other embodiments, the distal end 534 may be blunt, angled, or rounded.

In the pictured embodiment, the catheter 515 is shaped and sized for insertion into a lumen of an AV access site and associated blood vessels such that a longitudinal axis LA of the catheter 515 aligns with a longitudinal axis of the vessel at any given position within the vessel lumen. In that regard, the straight configuration illustrated in FIG. 11 is for exemplary purposes only and in no way limits the manner in which the catheter 515 may curve in other instances. Generally, the catheter 515 may be configured to take on any desired arcuate profile when in the curved configuration. In one instance, the catheter 515 has an overall length from the proximal end 532 to the distal end 534 of at least 40 cm and in some embodiments, extending to 120 cm. Other lengths are also contemplated. In some instances, the catheter 515 has an external diameter D7 ranging from 1.9° F. (0.63 mm) to 4° F. (1.35 mm).

The catheter 515 is formed of a flexible material such as, by way of non-limiting example, high density polyethylene, polytetrafluoroethylene, Nylon, block copolymers of polyamide and polyether (e.g., PEBAX), polyolefin, polyether-ester copolymer, polyurethane, polyvinyl chloride, combinations thereof, or any other suitable material for the manufacture of flexible, elongate catheters. In the pictured embodiment, the catheter 515 is connected at the proximal end 532 to an adapter 542, which is configured to couple the catheter to another medical device at a proximal port 544 and/or through an electrical connection 546. Various medical devices that may be coupled to the catheter 515 at the proximal port 544 include, by way of non-limiting example, a storage vessel, a disposal vessel, a vacuum system, a syringe, an infusion pump, and/or an insufflation device. In the pictured embodiment, the catheter is coupled to the PIM 122 by the electrical connection 546. Various other devices that may be coupled to the catheter 515 by the electrical connection 546 include, by way of non-limiting example, an energy generator (e.g., an ultrasound generator), a power source, the computer system 125, and/or the IVUS console 525.

It should also be appreciated that the delivery catheter 515 depicted herein is not limited to any particular type of catheter, and includes all ultrasonic or other imaging catheters known to those skilled in the art. For example, a catheter having a single transducer adapted for rotation or oscillation as well as a catheter having an array of transducers circumferentially positioned around the catheter are both within the spirit and scope of the present invention. Thus, in some embodiments, the transducer 540 may be a single element, mechanically-rotated ultrasonic device having a frequency of approximately 45 MHz. In other embodiments, the transducer 540 may comprise an array of transducers circumferentially positioned to cover 360 degrees, and each transducer may be configured to radially acquire radio frequency data from a fixed position on the catheter.

The computer device 125, which includes the processor 130 and the memory 132, utilizes the IVUS data to produce an IVUS image of the intravascular environment surrounding the transducer according to methods well known to those skilled in the art. Because different types and densities of tissue and other material absorb and reflect the ultrasound pulse differently, the reflected IVUS data can be used to image the vessel and the surrounding tissue and fluid. Multiple sets of IVUS data are typically gathered from multiple locations within a vascular object (e.g., by moving the transducer linearly through the vessel). These multiple sets of data can then be used to create a plurality of two-dimensional (2D) images or one three-dimensional (3D) image. In some embodiments, the system 500 may include an image analysis tool used after the acquisition of IVUS images. Intraluminal imaging may be done as an initial step to help determine the best applicable therapy, to observe a therapeutic measure in real-time, or as a later step to assess the results of a given therapy.

In some embodiments, the computer device 125 processes image data received from the catheter 515 and sensed data received from the sensor assembly 202 from the AV access site and surrounding vasculature. In such embodiments, the display 520 and/or the PIM 122 may display the processed data in a variety of forms, including by way of non-limiting example, graphical, two-dimensional, 3-dimensional, black-and-white, and color views. In some embodiments, the display 520 may display the blood pressure and/or blood flow information as a color overlay on the IVUS images. For example, in some embodiments, the display 520 may have similar features to those of the Chromaflo® Imaging and/or the ComboMap® Pressure and Flow System sold by Volcano Corporation of San Diego, Calif.

In some embodiments, the delivery catheter 515 may include radiopaque or inked markers to assist in the positioning and visualization of the catheter within the patient's AV access site and associated vasculature. For example, FIG. 12 illustrates an IVUS catheter 550, which is substantially identical to the delivery catheter 515 except for the differences described herein. The IVUS catheter 550 comprises an elongated, flexible tubular member or body 552 including a central lumen 555 that allows the passage of contents from a proximal end 560 through a distal end 565 of the catheter 550. A radiopaque marker coil 570 is positioned at a distal portion 575 of the body 552. The marker coil 570 provides radiopaque markers in the form of tightly wound sections 580 separated by loosely wound sections 585 to assist in positioning the transducer 540 within a patient's AV access site and associated vasculature and obtaining accurate visualization and measurements of the patient's AV access site and associated vasculature. In some instances, the processor 130 may coregister IVUS images with angiography data using length and positional measurements indicated by the radiopaque markers.

As shown in FIG. 13, the marker coil 570 is formed of a single length of radiopaque material that has been wound into areas of varying pitch. The tightly wound sections 580 form areas of greater radiopacity while loosely wound sections 160 form areas of less radiopacity. Thus, the tightly wound sections 580 effectively form radiopaque markers separated from each other by the loosely wound sections 585. In some instances, the imaging device 540 may be used to determine the morphology and pathology of a target lesion within a patient's anatomy (e.g., a stenosis or thrombosis within an AV access site and/or vessel). The radiopaque tightly wound sections 580 allow for the accurate localization of the sensor assembly 202 as well as the accurate localization and measurement of such a lesion. In some embodiments, the delivery catheter 515 includes a marker coil comprising features of the marker coil disclosed in U.S. Provisional Patent Application No. 61/692,603, entitled “Device, System, and Method Utilizing a Radiopaque Coil for Anatomical Lesion Length Estimation,” filed on Aug. 23, 2012 with inventor Stigall, which is hereby incorporated by reference in its entirety.

As illustrated in FIG. 14, in some embodiments, the system 500 utilizes a therapeutic balloon catheter 600 to treat occlusions and obstructions within the patient's AV access site and surrounding vasculature, such as stenosis and thrombosis. The balloon catheter 600 is substantially identical to the delivery catheter 515 except for the differences noted herein. The balloon catheter 600 includes a sensor assembly 605, which may include any number and type of sensors, including without limitation a pressure sensor, a flow sensor, a temperature sensor, or an imaging device. The catheter 515 includes a balloon assembly 610 with an outer sleeve 615 and an inner sleeve 620. The balloon assembly 610 is joined to a proximal shaft 624 of the catheter 600 through a proximal junction 626. Additionally, the balloon assembly 610 is joined to a mid-shaft 628 of the catheter 600 through a distal junction 630. In the illustrated embodiment, the mid-shaft 628 extends between the balloon assembly 610 and the sensor assembly 605. An inner member 632 defining a guide wire lumen 634 runs from a distal end 636 of the catheter, through the interior of the proximal shaft 624, the balloon assembly 610, and the mid shaft 628, to at least the proximal end of the balloon assembly 610. The proximal shaft 624 connects the balloon assembly 610 to a pressurized fluid system while a connection medium, such as electrical conductors or optical fibers, extending within the proximal shaft 624 connect the sensing device 116 to a processing systems (not shown) at the proximal end of the catheter 600. In some embodiments, the connection medium extends through the entire length of the balloon assembly 610 and joins the sensor assembly 605. In some instances, the sensor assembly 605 comprises an IVUS imaging device, such as an ultrasound transducer. The inner member 632 defines the guidewire lumen 634, which is sized to receive a sensor wire (i.e., the sensor wire 510 shown in FIG. 11) and allow reciprocal motion of the sensor wire along the longitudinal axis of the inner member. In some embodiments, the balloon catheter 600 includes features disclosed in U.S. Provisional Patent Application No. 61/734,825, entitled “High Pressure Therapeutic and Imaging Catheter,” filed on Dec. 7, 2012 with inventor Stigall, which is hereby incorporated by reference in its entirety.

FIG. 15 is a diagrammatic illustration of a cross-sectional view of the AV access site 700 (i.e., an AV graft or AV fistula) connecting an artery 705 and vein 710. The AV access site 700 has a wall 712 and a lumen 714. The artery 705 has an arterial wall 720 and an arterial lumen 722. The vein has a venous wall 724 and a venous lumen 726. The blood flow through the AV access site 700, the artery 705, the collaterals 730, and the vein 710 are indicated by the arrows.

FIGS. 16-21 show a method of inserting the delivery catheter 515 and the sensor wire 510 into an AV access site 700 to evaluate the functionality of the AV access site according to one embodiment of the present disclosure. FIG. 16 illustrates the sensor wire 510 and the delivery catheter 515 positioned within the arterial lumen 722 at a position above the entrance to the collaterals 730. The user can access the area of interest neighboring the AV access site using standard techniques known in the art employing a needle, a guidewire (e.g., the sensor wire 510), radiopaque markers, fluoroscopy, and the delivery catheter. In another instance, the user may access the area of interest by navigating the patient's vasculature using guided IVUS imagery via the delivery catheter 515. In operation, the distal end 534 of the catheter 515 is maneuvered through the vasculature until the transducer 540 reaches an intravascular position of interest in preparation to obtain IVUS data of the surrounding vascular tissue and fluid. In some instances, the user may advance the sensor wire 510 into the circulation past the distal end 534 of the catheter 515. In other instances, the user may advance the sensor wire 510 and the catheter 515 together. The appropriate positioning of the sensor assembly 202 may be confirmed by IVUS imaging via the ultrasound transducer 540 on the distal catheter 515 or may be confirmed via radiopaque markers. In some instances, the processor 130 may coregister IVUS images with angiography data using length and positional measurements indicated by radiopaque markers on the delivery catheter 515, and confirm appropriate positioning of the catheter.

Once positioned, the ultrasound transducer 540 may gather IVUS data, including characteristics, parameters, and measurements about the blood vessel and its contents, such as, by way of non-limiting example, data about the position of the sensor wire and data about the shape of the blood vessel, its density, and its composition. Specifically, the transducer 540 is pulsed to acquire echoes or backscattered signals reflected from the vascular tissue. Once appropriately positioned within the artery 705, the processor 130 and/or the user can activate the sensor assembly 202 to obtain the desired measurements, including by way of non-limiting example the blood pressure, flow rate, and temperature. Such measurements reflect the patient's circulatory function above the level of the AV access site.

FIG. 17 illustrates the sensor wire 510 and the delivery catheter 515 positioned within the arterial lumen 722 at a position below the entrance to the collaterals 730 and above the entrance to the AV access site 700. In the pictured embodiment, the user advances the sensor wire 510 into the circulation past the distal end 534 of the catheter 515. The appropriate positioning of the sensor assembly 202 may be confirmed by IVUS imaging via the ultrasound transducer 540 on the distal catheter 515 or may be confirmed via radiopaque markers. Once appropriately positioned within the artery 705, the processor 130 and/or the user can activate the sensor assembly 202 to obtain the desired measurements, including by way of non-limiting example the blood pressure, flow rate, and temperature.

FIG. 18 illustrates the sensor wire 510 and the delivery catheter 515 positioned within the arterial lumen 722 at a position below the entrance to the AV access site 700 and above the entry of the collateral blood flow into the arterial lumen 722. In the pictured embodiment, the user advances the catheter 515 to the entrance of the AV access site 700 and advances the sensor wire 510 into the circulation past the distal end 534 of the catheter 515. The appropriate positioning of the sensor assembly 202 may be confirmed by IVUS imaging via the ultrasound transducer 540 on the distal catheter 515 or may be confirmed via radiopaque markers. Once appropriately positioned within the artery 705, the processor 130 and/or the user can activate the sensor assembly 202 to obtain the desired measurements, including by way of non-limiting example the blood pressure, flow rate, and temperature. In some instances, the sensor wire 510 may be inserted into the collaterals 730 to obtain blood flow and pressure measurements within the collaterals.

FIG. 19 illustrates the sensor wire 510 positioned within the arterial lumen 722 at a position below the entrance to the AV access site 700 and below the entry of the collateral blood flow into the arterial lumen 722. In the pictured embodiment, the user advances the catheter 515 to the entrance of the AV access site 700 and advances the sensor wire 510 into the circulation past the distal end 534 of the catheter 515. The appropriate positioning of the sensor assembly 202 may be confirmed by IVUS imaging via the ultrasound transducer 540 on the distal catheter 515 or may be confirmed via radiopaque markers. Once appropriately positioned within the artery 705, the processor 130 and/or the user can activate the sensor assembly 202 to obtain the desired measurements, including by way of non-limiting example the blood pressure, flow rate, and temperature. Such measurements reflect the effect of the diversion of blood flow through the AV access site on the remaining circulation to the tissues distal to the AV access site.

FIG. 20 illustrates the sensor wire 510 and the delivery catheter 515 positioned within the AV access site 700. In the pictured embodiment, the user advances the catheter 515 to the entrance of the AV access site 700 and advances the sensor wire 510 into the AV access site past the distal end 534 of the catheter 515. By keeping the position of the imaging catheter 515 stationary at the entrance to the AV access site, the user to can efficiently advance the sensor wire 510 into the AV access site by completely retracting the sensor wire into the lumen 530 of the catheter 515 and turning it into the AV access site (thus obviating the need to relocate the entrance to the AV access site). The appropriate positioning of the sensor assembly 202 may be confirmed by IVUS imaging via the ultrasound transducer 540 on the distal catheter 515 or may be confirmed via radiopaque markers. Once appropriately positioned within the artery 705, the processor 130 and/or the user can activate the sensor assembly 202 to obtain the desired measurements, including by way of non-limiting example the blood pressure, flow rate, and temperature.

FIG. 21 illustrates the sensor wire 510 and the delivery catheter 515 positioned within the venous lumen 726 at a position beyond the exit of the AV access site 700. In the pictured embodiment, the user advances the catheter 515 into AV access site 700 and advances the sensor wire 510 into the venous circulation past the distal end 534 of the catheter 515. The appropriate positioning of the sensor assembly 202 may be confirmed by IVUS imaging via the ultrasound transducer 540 on the distal catheter 515 or may be confirmed via radiopaque markers. Once appropriately positioned within the artery 705, the processor 130 and/or the user can activate the sensor assembly 202 to obtain the desired measurements, including by way of non-limiting example the blood pressure, flow rate, and temperature.

As the catheter 515 navigates through the AV access site 700 and the surrounding vasculature, the IVUS transducer 540 can gather imaging data about the structure of the vessels and the AV access site to allow the processor to evaluate for the presence of vessel pathology, such as, by way of non-limiting example, stenosis and thrombosis. Once the processor 130 and/or the user has gathered the necessary data from the sensor assembly 202 and the IVUS transducer 540, the processor 130 may analyze the data to determine evaluate for the presence of complications associated with AV access sites such as, by way of non-limiting example, stenosis, thrombosis, DASS, and infection.

As described above, dialysis-associated steal syndrome (“DASS”) describes vascular insufficiency resulting from the diversion of blood flow through a vascular access site. In particular, to evaluate for the presence of DASS, the processor can compare the flow and pressure measurements obtained above within the arterial circulation above the entrance to the AV access site 700 and those measurements obtained below the entrance to the AV access site. If the comparison indicates inadequate perfusion to the tissue distal of the AV access site 700, then the display 520 and/or the PIM 122 can indicate the possibility of DASS to the user. In some instances, the processor 130 compares the sensed measurements to control values stored within the memory 132 and makes a determination as to the presence or absence of DASS and, if present, the extent of DASS. In some instances, the memory 132 stores the measured values obtained from a patient over time (e.g., from multiple dialysis appointments). In some instances, the memory 132 stores predetermined measurement gradients or ratios (i.e., comparing measurements taken from one vascular location to another) to indicate different clinical scenarios. For example, a first stored measurement gradient comparing the pressure and flow measurements above the level of the collateral circulation (as shown in FIG. 16) to the pressure and flow measurements below the level of the collateral circulation (as shown in FIG. 19) may indicate the presence of DASS. A different stored measurement gradient comparing the pressure and flow measurements within the AV access site (as shown in FIG. 20) to the pressure and flow measurements within the venous circulation (as shown in FIG. 26) may indicate the presence of venous outflow obstruction (e.g., stenosis or thrombosis). Correlation of the data from the sensor wire with the imaging data from the imaging catheter can assist the user in determining the location and morphology of the obstruction. After the processor 130 processes the sensed measurement data and the imaging data, the processor can determine if any access related complications are present, and, if so, which particular access associated complications are present. The display 520 and/or the PIM 122 can display the these determinations to the user.

FIG. 22 is a diagrammatic illustration of a cross-sectional view of the AV access site 800 (i.e., an AV graft or AV fistula) connecting an artery 805 and vein 810. The AV access site 800 has a wall 812 and a lumen 814. The artery 805 has an arterial wall 820 and an arterial lumen 822. The vein has a venous wall 824 and a venous lumen 826. The blood flow through the AV access site 800, the artery 805, the collaterals 830, and the vein 810 are indicated by the arrows. The AV access site 800 includes three stenotic segments 835, 840, and 845 at the venous anastomosis 820. In the pictured embodiment, the stenotic segments represent hyperplastic neointimal thickening of the vessel wall, which commonly occurs at the site of the venous anastomosis 820 and arterial anastomosis (e.g., 825). It should be understood that in some sections, the stenotic segments can collectively form an annular segment which resides along the entire circumference of the inner vessel and/or AV access site wall 812.

FIGS. 23-26 show a method of inserting the balloon catheter 600 and the sensor wire 510 into an AV access site 700 to both evaluate the functionality of the AV access site and treat the stenotic segments according to one embodiment of the present disclosure. It is important to note that all of the measurement and imaging activities described above in relation to FIGS. 16-21 may also be performed with the balloon catheter and sensor wire 510. In the pictured embodiment, the sensor wire 510 is positioned within the lumen 634 of the inner member 632 of the balloon catheter 600 (shown in FIG. 14). In FIG. 23, the balloon catheter 600, carrying the sensor wire 510, is positioned within the AV access site 700 at a position proximal to the stenosis. The user may have guided the balloon catheter 600 to the desired location using standard techniques known in the art employing a needle, a guidewire (e.g., the sensor wire 510), radiopaque markers, fluoroscopy, and/or the imaging device on the sensor assembly 605, as described above in relation to FIGS. 16-21. In operation, the distal end 636 of the catheter 600 is maneuvered through the vasculature until the transducer 605 (i.e., the sensor assembly 605) reaches an intravascular position of interest in preparation to obtain IVUS data of the surrounding vascular tissue and fluid. In some instances, the user may advance the sensor wire 510 into the circulation past the distal end 636 of the catheter 600. In other instances, the user may advance the sensor wire 510 and the catheter 515 together. The appropriate positioning of the sensor assembly 202 may be confirmed by IVUS imaging via the ultrasound transducer 605 on the balloon catheter 600 and/or may be confirmed via radiopaque markers.

During insertion of the catheter 600, the balloon assembly 610 is not inflated and maintains a low profile in an unexpanded condition. As the user advances the catheter 600 through the AV access site and the associated vasculature, the user can view the imaging data obtained by the ultrasound transducer 605 and the pressure and flow measurements obtained by the sensor assembly 202 to assess the functionality of the AV access site. The imaging data can inform the user if there is some type of lesion or injury or infection of the vessel walls 820, 824 or the wall 812 of the AV access site. The imaging data may also relay other vascular information about the AV access site and associated vessels, such as, by way of non-limiting example, the regularity or irregularity of the vessel walls and AV access site wall, the tortuosity and path of the AV access site, and the location and sizes of the collateral circulation. While the ultrasound transducer 605 is obtaining intravascular images, the sensor assembly 202 of the sensor wire 510 may be advanced through the distal end 636 of the catheter 600 to obtain pressure and flow measurements distal to the catheter. For example, in FIG. 23, as the balloon catheter 600 obtains intravascular images of the stenotic segments 835, 845, the sensor wire 510 has advanced into the vein 810 distal to the AV access site to measure various cardiovascular characteristics. Thus, the combined functionality of the sensor wire and the imaging catheter 600 allow for increased efficiency in the evaluation of AV access site functionality.

FIG. 24 illustrates the balloon assembly 610 positioned within the AV access site 800 and centered between the stenotic segments 835, 845. The images received from the ultrasound transducer 605 can be used to facilitate the placement of the balloon assembly 610 in relation to the stenotic segments. The stenotic segments have a proximal end and a distal end, as well as a length extending from the proximal end to the distal end. As the catheter 600 traverses the AV access site, a user can continue to image the stenotic segments as the catheter passes through the segments to obtain their dimensions and luminal contours (e.g., the intraluminal diameters of the AV access site 800 proximal, adjacent, and distal to the stenosis). In addition, the data received from the sensor assembly 202 may convey characteristics of blood flow through the stenosis to the user. Upon visualizing the stenotic segments 835, 845 and obtaining their various characteristics, the user can use this data to accurately advance the catheter 600 and center the balloon assembly 610 within the stenotic segments.

FIG. 25 illustrates the expansion of the balloon assembly 610 within the stenotic segments 835, 845 in the AV access site 800. After appropriately positioning the balloon assembly 610 within the stenotic segments, the user may inflate the balloon assembly to both relieve the obstruction caused by the stenosis. In some instances, the user may simultaneously expand a stent (not shown) to maintain the new patency of the AV access site 800.

FIG. 26 illustrates the deflation of the balloon assembly 610 and reassessment of the functionality of the AV access site. After reducing the obstruction, the user can deflate the balloon assembly and reassess the functionality of the AV access site by obtaining images from the ultrasound transducer 605 and intravascular pressure and flow measurements from the sensor assembly 202 of the sensor wire 510. In some instances, this necessitates advancement of retraction of the catheter 600 within the AV access site 800 and the surrounding vessels. In the pictured embodiment, these measurements would indicate to the user that further areas of obstruction (i.e., stenotic segment 840) remain, and the user can repeat the steps illustrated in FIGS. 23-26 to address these obstructions.

The devices, systems, and methods described herein offer the user a faster and more accurate approach to assessment of AV access site functionality by allowing the user to assess vascular pressure and flow characteristics of the access site and surround vessels and treat various complications of AV access sites in a single procedure. The devices, systems, and methods described herein can be particularly useful in patients having long-term AV access sites secondary to dialysis, chemotherapy, or liver disease.

It should be appreciated that while the exemplary embodiment is described in terms of an ultrasonic device, to render images of a vascular object, the present disclosure is not so limited. It should be noted that the catheter 515 depicted herein is not limited to a particular type of device, and includes any of a variety of imaging devices. Thus, for example, using backscattered data (or a transformation thereof) based on other sources of energy, such as electromagnetic radiation (e.g., light waves in non-visible ranges such as used in Optical Coherence Tomography, X-Ray CT, spectroscopy, etc.), to render images of any tissue type or composition (not limited to vasculature, but including other structures within a human or non-human patient) is within the spirit and scope of the present disclosure.

Persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. A method of evaluating an arteriovenous access site, the method comprising:
inserting a sensor into a vessel adjacent to the arteriovenous access site;
sensing with the sensor at least one of pressure, imaging and flow within the vessel adjacent to arteriovenous access site.

2. The method of claim 1, wherein the sensing includes taking a first pressure measurement within the vessel at a first location.

3. The method of claim 2, further including taking a second pressure measurement at a second position within the vessel, the first location being different than the second location.

4. The method of claim 3, further including comparing the first pressure with the second pressure to determine a pressure differential between the first location and the second location.

5. The method of claim 3, wherein the vessel forms at least a portion of the arteriovenous access site.

6. The method of claim 1, wherein the sensing includes taking a first flow measurement within the vessel at a first location.

7. The method of claim 1, wherein the sensing includes imaging a portion of the vessel.

8. The method of claim 7, wherein the imaging includes intravascular ultrasound imaging.

9. The method of claim 7, wherein the imaging includes intravascular optical coherence tomography (OCT) imaging.

10. The method of claim 1, wherein the sensing includes a plurality of one of pressure, flow, and imaging.

11. A system for evaluating an arteriovenous access site, the system comprising:
an access catheter sized to be received within a vessel forming at least a portion of an arteriovenous access site;
a pressure sensing guidewire moveable within the access catheter, the pressure sensor sending a pressure signal corresponding to the sensed pressure;
a processor configured to receive the pressure signal and evaluate the effectiveness arteriovenous access site based at least in part on the pressure signal; and
a graphical user interface configured to receive the output of the processor and provide a user with an indication of the effectiveness of the arteriovenous access site.

12. The system of claim 11, further including a second pressure sensor associated with the access catheter.

13. The system of claim 11, wherein the access catheter is substantially rigid.

14. The system of claim 11, further including an ultrasound sensor attached to one of the catheter or the guidewire.

15. The system of claim 11, further including an optical coherence tomography (OCT) element attached to one of the catheter or guidewire.

16. The system of claim 11, wherein the processor is further configured to receive imaging data for a portion of the vessel.

17. The system of claim 16, wherein the processor is further configured to process the imaging data for display on the graphical user interface.

18. The system of claim 16, wherein the imaging data is intravascular ultrasound (IVUS) data.

19. The system of claim 16, wherein the imaging data is intravascular optical coherence tomography (OCT) data.
